(19) United States
(12) Patent Application Publication
(10) Pub. No.: US 2018/0338951 A1
(43) Pub. Date: Nov. 29, 2018

(54) TREATMENTS FOR ARTERIAL STIFFENING, HYPERTENSION AND ANTI-AGING

(71) Applicant: The Board of Regents of the University of Oklahoma, Norman, OK (US)

(72) Inventor: Zhongjie Sun, Edmond, OK (US)

(21) Appl. No.: 15/985,154

(22) Filed: May 21, 2018

Related U.S. Application Data
(60) Provisional application No. 62/509,818, filed on May 23, 2017.

(51) Int. Cl. A61K 31/4045; A61P 3/10; A61P 9/12

(52) U.S. Cl. CPC A61K 31/4045; A61P 9/12; A61P 3/10

(57) ABSTRACT

Compositions and methods for the treatment and amelioration of arterial stiffness, hypertension, and/or arterial aging in a subject. In certain embodiments, the active agents of the compositions provide anti-aging treatments by causing arterial remodeling by decreasing collagen production and increasing elastin production in a subject. In certain embodiments, the active agents can be used to treat a subject having diabetes or a diabetes-related disease or condition, such as but not limited to, Type 1 diabetes mellitus (T1DM), Type 2 diabetes mellitus (T2DM), and hyperinsulenima (pre-diabetes). In certain embodiments, the active agents can be used to treat subjects having hypertension, aortic disease, cardiovascular disease, including heart failure (such as congestive heart failure), kidney disease, osteoporosis, Alzheimer's disease, infertility, and emphysema.

### CROSS-REFERENCE TO RELATED APPLICATIONS

This application claims priority to U.S. Provisional Application No. 62/509,818, filed on May 23, 2017, which is expressly incorporated herein by reference in its entirety.

### STATEMENT REGARDING FEDERALLY SPONSORED RESEARCH OR DEVELOPMENT

This invention was made with government support under Contract Numbers HL105302 and HL102074 from the National Institutes of Health. The government has certain rights in the invention.

### BACKGROUND

Millions of people suffer from heart attack and stroke each year. Arterial stiffness and hypertension are major risk factors for heart attack and stroke in the aged population. Arterial stiffness generally describes the elasticity or hardness of the arteries. The stiffness of arteries influences how hard the heart has to work to pump blood through the body. Hypertension generally refers to abnormally high blood pressure. Blood pressure is the force of blood pushing against the walls of arteries. Over time, arterial stiffness and hypertension can weaken and damage the cardiovascular system leading to heart attack and stroke.

Current antihypertensive drugs are primarily designed to reduce peripheral resistance and are not adequate to alter the pathological process of arterial stiffening or arterial stiffness-related hypertension. Peripheral resistance is the resistance of the arteries to blood flow. As arteries constrict, peripheral resistance increases and as arteries dilate, peripheral resistance decreases. Peripheral resistance is determined by factors such as: (i) autonomic activity, whereas sympathetic activity constricts peripheral arteries; (ii) pharmacologic agents, such as vasoconstrictor drugs which increase peripheral resistance and vasodilator drugs which decrease peripheral resistance; and (iii) blood viscosity, whereas increased viscosity increases peripheral resistance.

DNA demethylation is a physiological process that maintains transcriptional activity of genes. An increase in methylation in the promoter region of a gene diminishes the promoter activity and gene transcription. DNA methylation is increased with age and the prevalence of arterial stiffness and hypertension are also increased with age. Physiologically, an appropriate methylation level is maintained by the balanced methylase and demethylase activity.

The Klotho gene was originally identified as a putative aging-suppressor gene in mice that extended life span when overexpressed and induced a premature aging syndrome when disrupted. Subsequently, the Klotho gene was found to be involved in numerous aging-associated pathologies, including chronic kidney disease, diabetes, cancers, and cardiovascular diseases. A deficiency of the Klotho gene can cause arterial stiffness. Further, Klotho protein (also referred to herein as “Klotho”) levels decrease with age while the prevalence of arterial stiffness and hypertension increase with age. For example, at age 70 years, the serum level of Klotho protein in a human is only about one half of what it was at age 40 years. Moreover, the serum Klotho protein level is significantly decreased in humans with arterial stiffness and chronic kidney diseases.

Arterial stiffening is an independent predictor of cardiovascular outcomes, such as hypertension, myocardial infarction, cognitive decline in aging, stroke, and kidney diseases. However, the relationship between DNA methylation and aging-related arterial stiffening and hypertension has not been previously understood. For example, it has not been known if increased methylation could lead to arterial stiffening and hypertension, or if increased demethylation could attenuate arterial stiffening and hypertension. The present work addresses these questions.

### BRIEF DESCRIPTION OF THE DRAWINGS

Several embodiments of the present disclosure are hereby illustrated in the appended drawings. It is to be noted however, that the appended drawings only illustrate certain embodiments and are therefore not intended to be considered limiting of the scope of the present disclosure.

FIG. 1 graphically depicts the effects of the active agent (Compound H, a.k.a., Corn H) on arterial pulse wave velocity (PWV) and blood pressure (BP) in old mice in comparison to levels in untreated adult mice. (A) PWV as measured by 10-MHz Doppler probes. (B-E) pulse, systolic, diastolic, and mean BP, respectively, measured after 2 weeks of active agent treatment by the volume-pressure recording (“VPR”) tail-cuff method using a CODA 6 BP Monitoring System. Data are expressed as mean±standard error (SE) and analyzed by one-way ANOVA, n=6-7, *p<0.05, **p<0.01 vs adult mice; #p<0.05, ##p<0.01 vs old mice. Bars indicate the standard error of the mean (SEM).

FIG. 2 graphically depicts the effects of the active agent on DNA hypermethylation of Klotho gene in the kidney of old mice in comparison to levels in untreated adult mice. (A) DNA demethylase activity, n=6, with and without the active agent. (B) DNA methyltransferase activity, n=6, with and without the active agent. (C) DNA methylation index of Klotho gene, n=4, with and without the active agent. Data are expressed as mean±SE and analyzed by one-way ANOVA, *p<0.05, **p<0.01 vs adult mice; #p<0.05, ##p<0.01 vs old mice. Bars indicate the SEM.

FIG. 3 graphically depicts the effects of the active agent on full-length and secreted Klotho protein (“Klotho”) levels in the kidney and serum of old mice in comparison to levels in untreated adult mice. (A) western blot analysis of Klotho in kidney, n=4. (B) western blot analysis of Klotho in Serum, n=4. (C) Klotho mRNA expression in kidney, n=4. Data are expressed as mean±SE and analyzed by one-way ANOVA, *p<0.05, **p<0.01 vs adult mice; #p<0.05, ##p<0.01 vs old mice. Bars indicate the SEM.

FIG. 4A graphically depicts the effects of the active agent on the accumulation of collagen and degeneration of elastin in the aorta of old mice in comparison to levels in untreated adult mice. The histological and immunohistochemical staining results of collagen-1 and elastin are indicated by arrows, n=5. The active agent decreases the amount of collagen and increases the amount of elastin in old mice. Data are expressed as mean f SE and analyzed by one-way ANOVA, *p<0.05, **p<0.01 vs adult mice; #p<0.05, ##p<0.01 vs old mice. Bars indicate the SEM.

FIG. 4B graphically depicts western blot analysis of the effects of the active agent on collagen- and elastin in old mice, in comparison to levels in untreated adult mice, n=4. The western blot analysis confirms that the active agent decreases the amount of collagen and increases the amount of elastin in old mice. Data are expressed as mean±SE and analyzed by one-way ANOVA, *p<0.05, **p<0.01 vs adult mice; #p<0.05, ##p<0.01 vs old mice. Bars indicate the SEM. Tubulin is used as a control protein.

FIG. 5A graphically depicts by zymogram PAGE that the active agent significantly reduced the increases in arterial matrix metalloproteinase-2 (MMP-2) and matrix metalloproteinase-9 (MMP-9) activity and expression in old mice in comparison to levels in untreated adult mice. Data are expressed as mean±SE and analyzed by one-way ANOVA, n=4, *p<0.05, **p<0.01 vs adult mice; #p<0.05, ##p<0.01 vs old mice. Bars indicate the SEM.

FIG. 5B graphically depicts by western blot analysis that the active agent significantly reduced MMP-2 and MMP-9 expression in old mice in comparison to levels in untreated adult mice. Data are expressed as mean±SE and analyzed by one-way ANOVA, n=4, *p<0.05, **p<0.01 vs adult mice; #p<0.05, ##p<0.01 vs old mice. Bars indicate the SEM.

FIG. 6 graphically depicts by western blot analysis the effects of the active agent on levels of arterial transforming growth factor-PI (TGF-3), transforming growth factor-β3 (TGF-β3), runt-related transcription factor 2 (RUNX2) and alkaline phosphatase (ALP) expression in old mice in comparison to untreated adult mice. (A) western blot analysis of TGF-β1, TGF-β3, RUNX2 and ALP. (B) quantification of TGF-β1 protein levels. (C) quantification of TGF-β3 protein levels. (D) quantification of RUNX2 protein levels. (E) quantification of ALP1 protein levels. Data are expressed as mean±SE and analyzed by one-way ANOVA, n=4, *p<0.05, **p<0.01 vs adult mice; #p<0.05, ##p<0.01 vs old mice. Bars indicate the SEM.

FIG. 7A graphically depicts by western blot analysis the effects of the active agent on the Silent information regulator T1 enzyme (SirTI) activity and on its substrate acetyl p53 tumor suppressor protein (Ace-p53). The active agent increased SirTI activity in the aorta of old mice. Data are expressed as mean±SE and analyzed by one-way ANOVA, n=4, *p<0.05, **p<0.01 vs adult mice; #p<0.05, ##p<0.01 vs old mice. Bars indicate the SEM.

FIG. 7B graphically depicts by western blot analysis the effects of the active agent on AMP-activated protein kinase (AMPK) and phospho-AMP-activated protein kinase (p-AMPK). Expression of both p-AMPK and AMPK was increased by the active agent in old mice. Data are expressed as mean±SE and analyzed by one-way ANOVA, n=4, *p<0.05, **p<0.01 vs adult mice; #p<0.05, ##p<0.01 vs old mice. Bars indicate the SEM.

FIG. 7(C) graphically depicts by western blot analysis the effects of the active agent on endothelial nitric oxide synthase (eNOS) and phospho-endothelial nitric oxide synthase (p-eNOS). Expression of both p-eNOS and eNOS was increased by the active agent in old mice. Data are expressed as mean±SE and analyzed by one-way ANOVA, n=4, *p<0.05, **p<0.01 vs adult mice; #p<0.05, ##p<0.01 vs old mice. Bars indicate the SEM.

FIG. 8 graphically depicts by western blot analysis that the active agent did not affect MMP2, MMP9, TGFβ1, and TGFβ3 expression in mouse vascular aortic smooth muscle cells (MOVAS). MOVAS were treated with active agent, Klotho free (KL (−)) medium, and/or secreted Klotho (SKL) for 16 h and then harvested for western blot analysis. Data is expressed as mean±SE and analyzed by one-way ANOVA, n=4, *p<0.05, **p<0.01 vs regular medium; #p<0.05, ##p<0.01 vs KL (−) medium. Bars indicate the SEM.

FIG. 9 graphically depicts by western blot that the active agent did not affect microtubule-associated protein light chain 3 (LC3) expression in aortas of old mice (A), hearts of old mice (B), or kidneys of old mice (C). Data is expressed as mean f SE and analyzed by one-way ANOVA, n=4, *p<0.05, **p<0.01 vs adult mice; #p<0.05, ##p<0.01 vs old mice. Bars indicate the standard error of the mean (SEM).

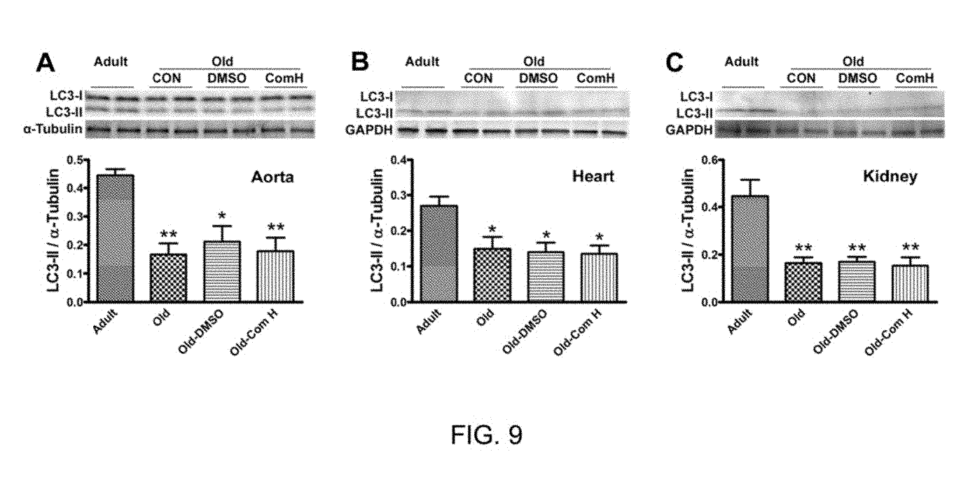

### DETAILED DESCRIPTION

The present disclosure describes methods and compositions comprising N-phenyl-1H-indole-3-carboxamide derivatives (also referred to herein as “active agents”), for example, N-halophenyl-1H-indole-3-carboxamides for the treatment of diseases and conditions such as, but not limited to, arterial stiffness, hypertension, and anti-aging effects, in a subject.

Before further describing various embodiments of the compounds, compositions and methods of the present disclosure in more detail by way of exemplary description, examples, and results, it is to be understood that the compounds, compositions, and methods of present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning; and the embodiments and examples are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications and equivalents apparent to those having ordinary skill in the art are included within the scope of the present disclosure.

All of the compounds, compositions, and methods of application and use thereof disclosed herein can be made and executed without undue experimentation in light of the present disclosure. Thus, while the compounds, compositions, and methods of the present disclosure have been described in terms of particular embodiments, it will be apparent to those of skill in the art that variations may be applied to the compounds, compositions and/or methods and in the steps or in the sequence of steps of the methods described herein without departing from the concept, spirit, and scope of the inventive concepts.

All patents, published patent applications, and non-patent publications mentioned in the specification or referenced in any portion of this application are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the term “about” is used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately”, where used herein when referring to a measurable value such as an amount, a temporal duration, and the like, is meant to encompass, for example, variations of ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment.

The term “pharmaceutically acceptable” refers to compounds and compositions which are suitable for administration to humans and/or animals without undue adverse side effects such as toxicity, irritation and/or allergic response commensurate with a reasonable benefit/risk ratio. The compounds of the present disclosure may be combined with one or more pharmaceutically-acceptable excipients, including carriers, vehicles, and diluents which may improve solubility, deliverability, dispersion, stability, and/or conformational integrity of the compounds or conjugates thereof. As used herein a “pharmaceutically acceptable carrier” is a pharmaceutically acceptable solvent, suspending agent, vehicle, or diluent for delivering the active agents of the present disclosure to a subject The carrier may be liquid or solid and is selected with the planned manner of administration in mind. Examples of pharmaceutically acceptable carriers that may be utilized with the active agents disclosed herein include but are not limited to polyethylene glycol (PEG) of various molecular weights, liposomes, ethanol, dimethyl sulfoxid (DMSO), aqueous buffers, oils, and combinations thereof.

As used herein, “pure,” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

Non-limiting examples of animals within the scope and meaning of this term include dogs, cats, rats, mice, guinea pigs, chinchillas, horses, goats, cattle, sheep, zoo animals, Old and New World monkeys, non-human primates, and humans.

“Treatment” refers to therapeutic treatments. “Prevention” refers to prophylactic or preventative treatment measures or reducing the onset of a condition or disease. The term “treating” refers to administering the composition to a subject for therapeutic purposes and/or for prevention. Non-limiting examples of modes of administration include oral, topical, retrobulbar, subconjunctival, transdermal, parenteral, subcutaneous, intranasal, intramuscular, intraperitoneal, intravitreal, and intravenous routes, including both local and systemic applications. In addition, the compositions of the present disclosure may be designed to provide delayed, controlled, extended, and/or sustained release using formulation techniques which are well known in the art.

The term “topical” is used herein to define a mode of administration through an epithelial surface, such as but not limited to, a material that is administered by being applied externally or internally to a surface.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of an active agent of the present disclosure which is sufficient to exhibit a detectable therapeutic or treatment effect in a subject without excessive adverse side effects (such as substantial toxicity, irritation and allergic response) commensurate with a reasonable benefit/risk ratio when used in the manner of the present disclosure. The effective amount for a subject will depend upon the subject's type, size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” means a detectable or measurable improvement in a subject's condition or symptom thereof. A detectable or measurable improvement includes a subjective or objective decrease, reduction, inhibition, suppression, limit or control in the occurrence, frequency, severity, progression, or duration of the condition, or an improvement in a symptom or an underlying cause or a consequence of the condition, or a reversal of the condition. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a condition, or consequences of the condition in a subject.

A decrease or reduction in worsening, such as stabilizing the condition, is also a successful treatment outcome. A therapeutic benefit therefore need not be complete ablation or reversal of the condition, or any one, most or all adverse symptoms, complications, consequences or underlying causes associated with the condition. Thus, a satisfactory endpoint may be achieved when there is an incremental improvement such as a partial decrease, reduction, inhibition, suppression, limit, control or prevention in the occurrence, frequency, severity, progression, or duration, or inhibition or reversal of the condition (e.g., stabilizing), over a short or long duration of time (e.g., seconds, minutes, hours).

In at least certain non-limiting embodiments, the present disclosure includes compositions and methods of treating a subject for arterial stiffness and/or hypertension, and/or aging effects, with an N-phenyl-1H-indole-3-carboxamide derivative. In certain embodiments the N-phenyl-1H-indole-3-carboxamide derivative is at least one N-halophenyl-1H-indole-3-carboxamide selected from the group: N-chlorophenyl-1H-indole-3-carboxamide, N-fluorophenyl-1H-indole-3-carboxamide, N-bromophenyl-1H-indole-3-carboxamide, and N-iodophenyl-1H-indole-3-carboxamide, or pharmaceutically-acceptable salts thereof, wherein the halogen is on the 2, 3, 4, 5, or 6-carbon position on the phenyl ring.

In certain embodiments, the N-halophenyl-1H-indole-3-carboxamide active agent is an N-(2-halophenyl)-1H-indole-3-carboxamide, or a pharmaceutically-acceptable salt thereof, having Formula I:

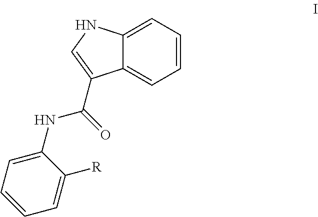

wherein R is selected from the group consisting of chlorine (Cl), fluorine (F), bromine (Br) and iodine (I).

In certain embodiments, the N-halophenyl-1H-indole-3-carboxamide active agent, or pharmaceutically-acceptable salt thereof, may have a halogen on two or more of the carbons of the phenyl ring, such as on two, three, four, or five carbons selected from the 2, 3, 4, 5, and 6-carbon positions of the phenyl ring, wherein the two or more halogens may be selected from Cl, F, Br, and I, and combinations thereof. The two or more halogens may be the same (e.g., both Cl) or different (e.g., Cl and F).

In at least one embodiment, the present disclosure is directed to a method of treating hypertension and/or arterial aging in a subject in need of such therapy, comprising: administering to the subject an effective amount of a composition comprising an N-halophenyl-1H-indole-3-carboxamide, or a pharmaceutically-acceptable salt thereof, wherein the N-halophenyl-1H-indole-3-carboxamide has a halogen on at least one of the 2, 3, 4, 5, and 6-carbon positions of the phenyl ring, wherein the halogen is selected from the group consisting of chlorine (Cl), fluorine (F), bromine (Br), and iodine (I). The N-halophenyl-1H-indole-3-carboxamide or pharmaceutically-acceptable salt thereof may be represented by Formula I above, wherein R in Formula I is selected from the group consisting of Cl, F, Br, and I. For example, R may be Cl.

In at least one embodiment, the present disclosure is directed to a method of treating arterial aging in a subject in need of such therapy, comprising administering to the subject an effective amount of a compound that increases arterial elastin production, the compound comprising an N-halophenyl-1H-indole-3-carboxamide, or a pharmaceutically-acceptable salt thereof, wherein the N-halophenyl-1H-indole-3-carboxamide has a halogen on at least one of the 2, 3, 4, 5, and 6-carbon positions of the phenyl ring, wherein the halogen is selected from the group consisting of chlorine (Cl), fluorine (F), bromine (Br), and iodine (I). The N-halophenyl-1H-indole-3-carboxamide or pharmaceutically-acceptable salt thereof may be represented by Formula I above, wherein R in Formula I is selected from the group consisting of Cl, F, Br, and I. For example, R may be Cl. The compound may further decrease arterial collagen production.

As noted above, the present disclosure describes methods of administering compositions comprising active agents for the treatment of certain diseases and conditions, including but not limited to, arterial stiffness, hypertension, diabetes, and anti-aging effects, in a subject. Without wishing to be bound by theory, it is believed that the active agents alter the pathological process of these conditions and diseases by activating DNA demethylases and increasing expression of Klotho protein. Further, the active agents and methods of use thereof are also directed to methods of providing anti-aging treatments. Without wishing to be bound by theory, it is believed that the active agents cause arterial remodeling by decreasing collagen production and increasing elastin production in the arterial tissues of subject. The methods disclosed herein are therefore directed to (1) methods of treating and attenuating arterial stiffening and/or hypertension by altering the pathological process of arterial stiffening and hypertension, and (2) to methods of providing arterial remodeling by decreasing collagen production and increasing elastin production in arterial tissues thereby resulting in anti-aging effects in the subject. The active agents described herein may be used to treat any disease or condition characterized by arterial stiffening, and/or by loss of elastin, including but not limited to, hypertension, aortic disease, cardiovascular disease, including heart failure (such as congestive heart failure), diabetes, kidney disease, osteoporosis, Alzheimer's disease, infertility, and emphysema.

In certain embodiments, the present disclosure is directed to compositions and conjugates comprising the active agent, wherein the compositions and conjugates may be utilized in methods of treating arterial stiffening and hypertension and for providing anti-aging treatments for providing arterial remodeling, and for treating other diseases and conditions identified herein. The inventive concepts disclosed herein also kits that include said active agent and compositions and conjugates thereof.

The present disclosure is also directed to methods of administering an effective amount of active agent to a subject, wherein the effective amount of active agent is sufficient to cause activation of DNA demethylases and attenuation of aging-related arterial stiffening and hypertension, and/or decreasing collagen production and increasing elastin production in arterial tissues for anti-aging treatments for providing arterial remodeling.

Without wishing to be bound by theory it is believed that the active agent increases DNA demethylase activity and decreases methylation of the Klotho gene, thereby increasing expression of Klotho protein. The resulting increase in circulating levels of Klotho protein caused by administration of the active agent is thought to reduce accumulation of stiffer collagen and reduce degeneration of compliant elastin fibers. Further, the increased Klotho expression attenuates the aging increased activity and expression of MMP2 (arterial matrix metalloproteinase-2), MMP9 (arterial matrix metalloproteinase-9) and the aging increased expression of TGF-β1 (transforming growth factor-β1), TGF-β3 (transforming growth factor-β3), RUNX2 (runt-related transcription factor 2), and ALP (alkaline phosphatase) to inhibit arterial fibrosis and stiffness.

In certain embodiments, the methods of the present disclosure are directed to treating arterial stiffness and/or hypertension in a subject, such as a human or other mammal, which is in need of such therapy, by administering a therapeutically-effective amount of an active agent, as disclosed herein. Without wishing to be bound by theory it is believed that the active agent has its effect by increasing the activity of the klotho gene thereby causing an increase in the expression of Klotho protein which leads to a reduction in arterial collagen production and an increase in arterial elastin production, thereby, attenuating arterial stiffness and hypertension.

In certain embodiments, the methods of the present disclosure are directed to causing arterial remodeling (anti-aging) in a subject, such as a human or other mammal, which is in need of such therapy, by administering a therapeutically-effective amount of an active agent, as disclosed herein. Without wishing to be bound by theory it is believed that the active agent has its effect by increasing the activity of the klotho gene thereby causing an increase in the expression of Klotho protein which leads to a reduction in arterial collagen production and an increase in arterial elastin production, thereby causing arterial remodeling, and the attenuation of arterial stiffness and hypertension.

Moreover, as noted, administration of the active agents of the present disclosure cause an increase in production of Klotho protein. Klotho protein is known as a treatment for diabetes or diabetes-related diseases or conditions (e.g., see Published PCT application WO 2014/152993 A1, expressly incorporated herein by reference in its entirety). In certain embodiments therefore, the presently disclosed active agents, by virtue of their effect in causing enhanced production of Klotho protein, can be used to treat a subject having diabetes or a diabetes-related disease or condition, such as but not limited to, Type 1 diabetes mellitus (T1DM), Type 2 diabetes mellitus (T2DM), and hyperinsulenima (pre-diabetes). Diabetes-related diseases or conditions include, but are not limited to: obesity; peripheral arterial disease (PAD) of the arms, legs, and feet; foot ulcers; hypertension; diabetic neuropathy; diabetic retinopathy; diabetic kidney disease; ketoacidosis, and hyperosmolar hyperglycemic nonketotic syndrome (HHNS).

T2DM generally progresses through several phases from pre-diabetes to full-blown insulin dependence. As elevated glucose levels occur in the body, it will place a higher demand on insulin secreting pancreatic beta-cells to produce insulin and restore glucose homeostasis (or alleviate postprandial spikes). This increased demand to fold insulin leads to endoplasmic reticulum stress (ERS) in the beta cells. ERS can also result in phosphorylation of the insulin receptor, which attenuates insulin efficacy, causing insulin resistance and, as a result, further increases the demand for insulin production. As this ERS in the beta cells continues, it will eventually lead to a loss in beta cell mass, which has been observed in autopsies of T2DM individuals. Since beta cells do not appear to be regenerated in the pancreas, this loss in beta cell mass leads to the fully insulin-dependent phenotype of later stage T2DM patients.

Preservation of pancreatic beta cells thus maintains beta cell mass due to amelioration of ERS. The latter serves to prevent one of the most deleterious outcomes in T2DM, which is loss of beta cell mass. In regard to T1DM, amelioration of beta cell loss during the early stages of the disease helps preserve beta cell mass while treatments against beta cell-killing autoimmunity of T1DM are mounted. Therefore, in at least one embodiment, the present disclosure includes a method of preserving beta cell mass in a subject suffering from diabetes or a diabetic-related condition by administering to the subject at least one of the active agents described herein. The preservation of beta cell mass in the subject can be shown by an increase or stabilization in the amount of insulin production and/or C-peptide production in the subject (as measured by blood or urine tests). In one suitable assay, beta cell mass is indirectly calculated by determining the ratio of C-peptide-to-glucose following oral glucose ingestion, particularly as measured 15 minutes after glucose ingestion (Meier et al., Diabetes 2009; 58:1595-1603). Alternatively, beta cell mass preservation can be indirectly calculated by using the Homeostasis model assessment (HOMA) index (Matthews et al., Diabetologia 1985; 28: 412-419). Additionally, in at least one embodiment, the administration of an active agent disclosed herein results in a blood hemoglobin A1C value which is less than about 7% in the subject.

The active agents of the present disclosure may be administered to a subject by any method known in the art, including but not limited to, oral, topical, transdermal, parenteral, subcutaneous, intranasal, intramuscular, intraperitoneal, and intravenous routes, and including both local and systemic applications. The active agents may also include a pharmaceutically acceptable carrier, such as a solvent, suspending agent, or vehicle for delivering the compositions or conjugates to the subject. In addition, the active agents disclosed herein may be configured to provide delayed or controlled release using formulation techniques which are well known in the art.

The present disclosure is also directed to a pharmaceutical composition comprising an effective amount of an active agent in combination with a pharmaceutically acceptable carrier. As used herein the term “effective amount” refers to an amount of a biologically active molecule or conjugate or derivative thereof sufficient to exhibit a detectable therapeutic effect without undue side effects (such as toxicity, irritation and allergic response) commensurate with a reasonable benefit/risk evaluation when used in the matter of the present disclosure. As noted above, the therapeutic effect may include, for example, attenuating arterial stiffening and hypertension, and reducing arterial remodeling. As one of ordinary skill in the art will readily appreciate, the effective amount for a particular subject, such as an adult male, adult female, or child, will depend upon the type of subject, the subject's size and health, the nature and severity of the condition to be treated, the method of administration, the duration of treatment, the nature of any additional therapy or treatment employed, the specific formulations employed, and the like.

The term “gene” is used herein for simplicity to refer to a functional protein-, polypeptide-, or peptide-encoding DNA unit. As will be understood by those in the an, this functional term includes genomic sequences, cDNA sequences or combinations thereof. “Isolated substantially away from other coding sequences” means the gene of interest forms the significant part of the coding region of the DNA segment, and that the DNA segment does not contain other non-relevant large portions of naturally-occurring coding DNA, such as large chromosomal fragments or other functional genes or DNA coding regions. This refers to the DNA segment as originally isolated, and does not exclude genes or coding regions later added to, or intentionally left in, the segment by a person.

The term “polypeptide” means a molecule comprising a series of amino acids linked through amide linkages along the alpha carbon backbone. Modifications of the peptide side chains may be present, along with glycosylation, hydroxylation and the like. Additionally, other non-peptide molecules, including lipids and small molecule agents may be attached to the polypeptide.

The nucleic acid segments of the present disclosure, regardless of the length of the coding sequence itself, may be combined with other DNA sequences, such as promoters, polyadenylation signals, additional restriction enzyme sites, multiple cloning sites, epitope tags, poly histidine regions, other coding segments, and the like, such that their overall length may vary considerably. It is, therefore, contemplated that nucleic acid fragments of almost any length may be employed, with the total length preferably being limited by the ease of preparation and use in the intended recombinant DNA protocol.

Practice of the methods of the present disclosure may comprise administering to a subject an effective amount of the active agent in any suitable systemic and/or local formulation, in an amount effective to deliver the dosages listed herein, or other acceptable dosages as determined by the attending physician. An effective amount of an active agent of the present disclosure will generally contain sufficient active agent to deliver, in certain embodiments, from about 0.1 μg/kg to about 100 mg/kg (mass of active agent/body weight of the subject). Particularly, the composition will deliver about 0.5 μg/kg to about 50 mg/kg, and more particularly about 1 μg/kg to about 10 mg/kg. The dosage can be administered, for example but not by way of limitation, on a one-time basis, or administered at multiple times (for example but not by way of limitation, from one to five times per day, or once or twice per week), or continuously via a venous drip, depending on the desired therapeutic effect. In one non-limiting embodiment of a therapeutic method, the active agent is provided in an IV infusion in the range of from about 0.1 μg/kg to about 10 mg/kg of body weight once a day.

Administration of the active agent used in the pharmaceutical composition or to practice the method of the present disclosure can be carried out in a variety of conventional ways, such as, but not limited to, orally, by inhalation, rectally, or by cutaneous, subcutaneous, intraperitoneal, or intravenous injection. Oral formulations may be formulated such that the active agent passes through a portion of the digestive system before being released, for example it may not be released until reaching the small intestine, or the colon.

When an effective amount of the active agent is administered orally, it may be in the form of a solid or liquid preparations such as capsules, pills, tablets, lozenges, melts, powders, suspensions, solutions, elixirs or emulsions. Solid unit dosage forms can be capsules of the ordinary gelatin type containing, for example, surfactants, lubricants, and inert fillers such as lactose, sucrose, and cornstarch, or the dosage forms can be sustained release preparations. The pharmaceutical composition may contain a solid carrier, such as a gelatin or an adjuvant. The tablet, capsule, and powder may contain from about 0.05 to about 95% of the active substance compound by dry weight. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol, or polyethylene glycol. When administered in liquid form, the pharmaceutical composition particularly contains from about 0.005 to about 95% by weight of the active agent. For example, a dose of about 10 mg to about 1000 mg once or twice a day could be administered orally.

In another embodiment, the active agents described herein can be tableted with conventional tablet bases such as lactose, sucrose, and cornstarch in combination with binders, such as acacia, cornstarch, or gelatin, disintegrating agents such as potato starch or alginic acid, and a lubricant such as stearic acid or magnesium stearate. Liquid preparations are prepared by dissolving the active agent in an aqueous or non-aqueous pharmaceutically acceptable solvent which may also contain suspending agents, sweetening agents, flavoring agents, and preservative agents as are known in the art.

For parenteral administration, for example, the active agent of the present disclosure may be disposed in a physiologically acceptable pharmaceutical carrier and administered as either a solution or a suspension. Illustrative of suitable pharmaceutical carriers are water, saline, dextrose solutions, fructose solutions, ethanol, or oils of animal, vegetative, or synthetic origin. The pharmaceutical carrier may also contain preservatives and buffers as are known in the art.

When an effective amount of the active agent is administered by intravenous, cutaneous, or subcutaneous injection, the active agent may be in the form of a pyrogen-free, parenterally acceptable aqueous solution or suspension. The preparation of such parenterally acceptable solutions, having due regard to pH, isotonicity, stability, and the like, is well within the skill in the art. A particular pharmaceutical composition for intravenous, cutaneous, or subcutaneous injection may contain, in addition to the active agent, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present disclosure may also contain stabilizers, preservatives, buffers, antioxidants, or other additives known to those of skill in the art.

As noted, particular amounts and modes of administration can be determined by one skilled in the art. One skilled in the art of preparing formulations can readily select the proper form and mode of administration, depending upon the particular characteristics of the active agent selected, the condition to be treated, the stage of the condition, and other relevant circumstances using formulation technology known in the art, described, for example, in Remington: The Science and Practice of Pharmacy. 21^{st} ed.

Additional pharmaceutical methods may be employed to control the duration of action of the active agent. Increased half-life and/or controlled release preparations may be achieved through the use of polymers to conjugate, complex with, and/or absorb the active agent described herein. The controlled delivery and/or increased half-life may be achieved by selecting appropriate macromolecules (for example but not by way of limitation, polysaccharides, polyesters, polyamino acids, homopolymers polyvinyl pyrrolidone, ethylenevinylacetate, methylcellulose, or carboxymethylcellulose, and acrylamides such as N-(2-hydroxypropyl) methacrylamide), and the appropriate concentration of macromolecules as well as the methods of incorporation, in order to control release. The active agent may also be ionically or covalently conjugated to the macromolecules described above.

Possible methods useful in controlling the duration of action of the active agent by controlled release preparations and half-life is incorporation of the active agent into particles of a polymeric material such as polyesters, polyamides, polyamino acids, hydrogels, poly(lactic acid), ethylene vinylacetate copolymers, copolymer micelles of, for example, polyethylene glycol (PEG) and poly(I-aspartamide).

It is also possible to entrap the active agent in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles, and nanocapsules), or in macroemulsions. Such techniques are well known to persons having ordinary skill in the art.

When the active agent is to be used as an injectable material, it can be formulated into a conventional injectable carrier. Suitable carriers include biocompatible and pharmaceutically acceptable phosphate buffered saline solutions, which are particularly isotonic.

The active agent may be formulated in a composition that includes a sterile diluent, which may contain materials generally recognized for approximating physiological conditions and/or as required by governmental regulation. In this respect, the sterile diluent may contain a buffering agent to obtain a physiologically acceptable pH, such as sodium chloride, saline, phosphate-buffered saline, and/or other substances which are physiologically acceptable and/or safe for use. In general, the material for intravenous injection in humans should conform to regulations established by the Food and Drug Administration, which are available to those in the field. The pharmaceutical composition may also be in the form of an aqueous solution containing many of the same substances as described above.

The active agents of the present disclosure can also be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines, and substituted ethanolamines.

Several embodiments of the present disclosure, having now been generally described, will be more readily understood by reference to the following example, which is included merely for purposes of illustration of certain aspects and embodiments of the present disclosure, and is not intended to be limiting. Those skilled in the art will promptly recognize suitable and appropriate variations from the various compositions, compounds, components, procedures and methods described in the example.

### Example

The object of this example is to describe the effect of one embodiment of an active agent of the present disclosure on arterial stiffness and hypertension in a subject, such as aged (old) mice for example. In this example the active agent is N-(2-chlorophenyl)-1H-indole-3-carboxamide, but it is to be understood that the methods of the present disclosure are not to be limited to this particular compound. Other N-phenyl-1H-indole-3-carboxamide derivatives, including but not limited to other N-halophenyl-1H-indole-3-carboxamides can effect the same results. This example demonstrates that aging-related arterial stiffening and hypertension are partially attributed to increased DNA methylation. The active agent is shown to increase Klotho expression and attenuate arterial stiffening and hypertension in old mice. Thus, the active agent is an effective therapeutic agent for aging-related arterial stiffening and hypertension. Another use of the active agent is to cause a decrease in arterial collagen production and an increase in arterial elastin production thereby providing arterial remodeling which serves as an anti-aging treatment.

In this example, old (“aged”) mice (24-30 months of age) were treated with or without Compound H (15 mg/kg, IP daily) for 2 weeks while adult mice (12 months of age) without treatments were used as controls. Compound H in this non-limiting example is N-(2-chlorophenyl)-1H-indole-3-carboxamide

Pulse wave velocity (PWV), a direct measure of arterial stiffness, and blood pressure (BP) were increased significantly in aged mice. Notably, the active agent reversed the age-related increases in PWV and BP within 2 weeks of treatment. The active agent effectively increased secreted Klotho levels in both kidney and serum through increasing DNA demethylase activity to inhibit methylation of Klotho gene. Aging-related arterial stiffness was associated with accumulation of stiffer collagen and degeneration of compliant elastin fibers. In addition, the activity and expression of MMP2, MMP9, and the expression of TGF-β1, TGF-ββ, RUNX2 and ALP were increased in aortas of aged mice. These changes were attenuated by the active agent. Mechanistically, the silent information regulator T1 enzyme-AMP-activated protein kinase-endothelial nitric oxide synthase (SIRT1-AMPK-eNOS) pathway may also take part in the therapeutic effect of the active agent.

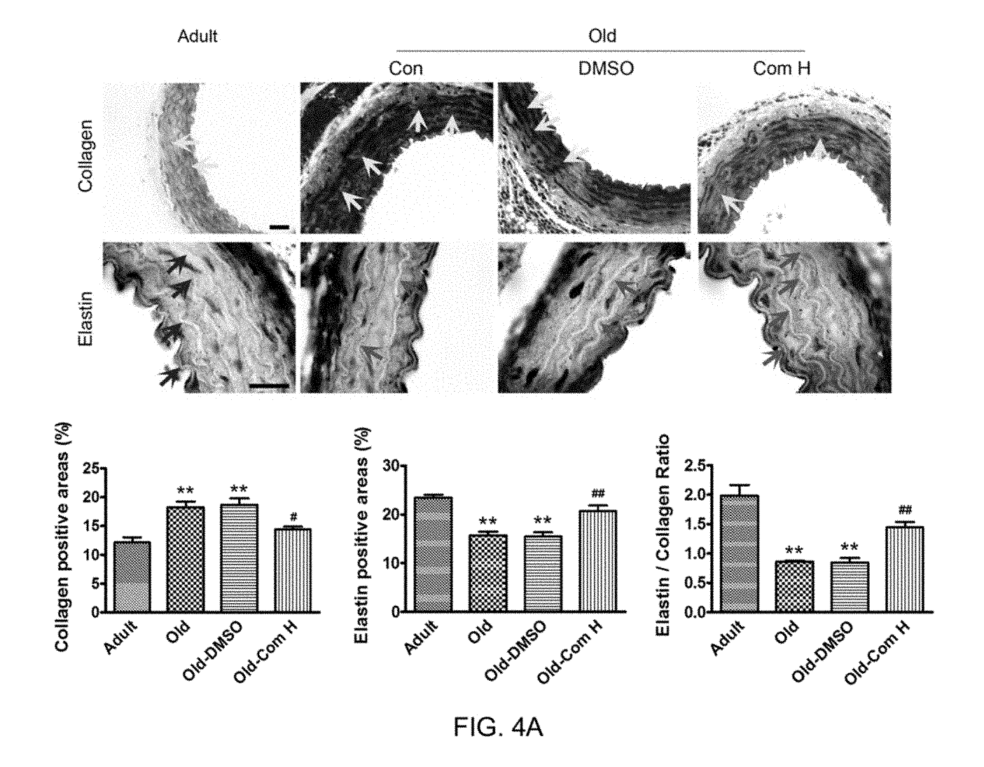

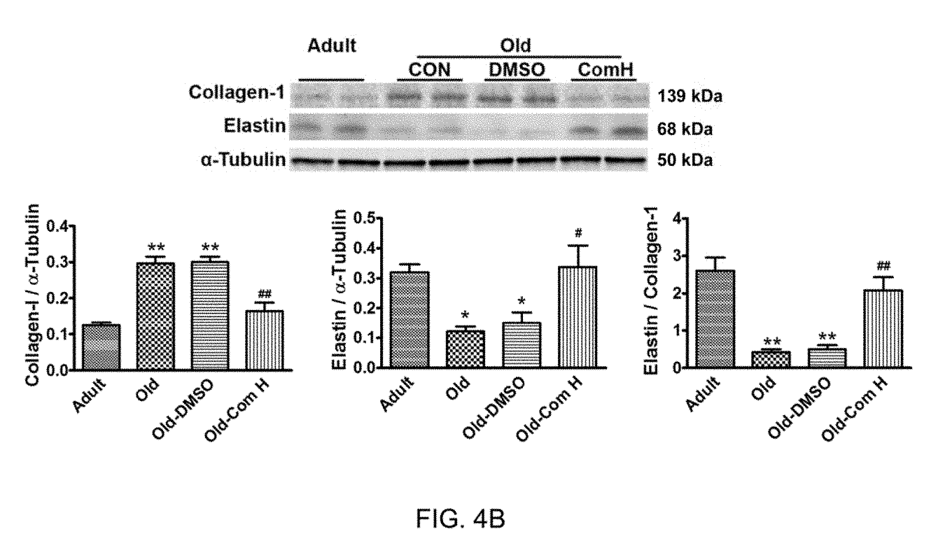

The active agent effectively attenuated the increases in PWV and BP in old mice, indicating that it is an effective therapeutic agent for arterial stiffness and hypertension. The active agent also effectively decreased collagen production and increased elastin production (as shown in FIG. 4B), indicating that it is effective in arterial remodeling for use as an anti-aging treatment.

Methods

Animal study protocols: This example was performed according to the guidelines of the National Institute of Health on the care and use of laboratory animals and approved by the Institutional Animal Care and Use Committee of University of Oklahoma Health Science Center. All mice were housed in cages at room temperatures (25±1° C.) and were provided with Purina laboratory chow (No. 5001) and tap water ad libitum. 6 adult mice (12 months) and 20 old mice (24-26 months) mice were used in this study. The old (aged) mice were randomly divided into three subgroups and each group had 6 or 7 mice. One subgroup received compound H (10 mg/kg/day, IP, Enamine LLC, Monmouth Jct., N.J.) and one group received an equal volume of DMSO (dimethyl sulfoxide, 5%) and served as a control. The third group received no treatment. Blood pressure was measured before and after treatment with the active agent at 1 week and 2 week. Pulse wave velocity (PWV) was measured after 2 weeks treatment with active agent. All animals were sacrificed and perfused transcardially with PBS under deep anesthesia (ketamine/xylazine, 90/10 mg, IP). The aortas were then quickly removed, washed, and cut into pieces for subsequent analyses.

Cell culture and treatment: MOVAS (ATCC&CRL-2797) is a continuous mouse aortic vascular smooth muscle cell line that has been demonstrated to retain a VSMC-like phenotype, including a spindle cell morphology and the expression of VSMC-specific markers such as smooth muscle α-actin and SM22-α. MOVAS were cultured in Dulbecco's modified Eagle's medium (DMEM) supplemented with 10% heat-inactivated fetal bovine serum (FBS) and 2 mM L-glutamine. After cells confluent, the media were switched to DMEM with or without 2% serum, and the cells were then treated with 10 μM active agent or 5 nM secreted Klotho for another 16 h then harvested for western blot analysis.

Measurement of Pulse Wave Velocity: Aortic PWV was measured as described previously. Briefly, mice were anesthetized under 2% isoflurane in a closed chamber anesthesia machine (SomnoSuite, Kent Scientific, Torrington, Conn.) for ˜1-3 min. Anesthesia was maintained via nose cone, and mice were secured in a supine position on a heating board (˜37° C.) to maintain body temperature. Velocities were measured with 6-mm crystal 20-MHz Doppler probes (Indus Instruments, Webster, Tex.) at the transverse aortic arch and ˜3.5 cm distal at the abdominal aorta and collected using Doppler signal processing workstation (Indus Instruments). Absolute pulse arrival times were indicated by the sharp upstroke, or foot, of each velocity waveform. Aortic PWV is then calculated as the quotient of the separation distance, assessed to the nearest half millimeter by engineering caliper and difference in absolute arrival times.

Measurement of Blood Pressure: Blood pressure was measured by the volume-pressure recording (VPR) tail-cuff method with slight warming (28° C.) but not heating of the tail using a CODA none-invasive blood pressure monitoring system (Kent Scientific). This method of measurement has been validated by using a telemetry system. Animals were gently handled and trained for the VPR tail-cuff measurement to minimize handling stress. No signs of stress were observed during BP measurements. The operator was also strictly trained for the measurement procedure. At least 20 stable cycle data were obtained for the result analysis for each measurement.

Histological and Immunohistochemical Staining: Thoracic aortas were quickly excised and placed in cold (4° C.) physiological saline solution. Three millimeter rings with perivascular tissue intact were removed from the thoracic aorta directly distal to the greater curvature of the aortic arch. Aorta rings were post-fixed in 4% paraformaldehyde, embedded in paraffin and sectioned at 5 μm thickness. Collagen was quantified by Masson's trichrome staining as described previously. The blue staining represented collagen deposition. A series of 5-10 sections of each mouse (5 mice per group) were examined and photographed using an Olympus BH-L microscope coupled with a digital color camera. Blue-stained collagen areas were quantified with ImageJ (NIH, Bethesda, Mass.) from 4-5 regions per section. The same threshold was used for each photo to make sure they are comparable. Elastin was assessed by immunohistochemical visualization. Briefly, sections are washed and incubated in primary antibody against elastin (1:50, Abcam, Cambridge, Mass., USA) or negative control (2.5% horse serum, Vector Labs) overnight and elastin was visualized using the appropriate secondary antibody. Finally, series of 5-10 sections were examined and photographed using an Olympus BH-L microscope coupled with a digital color camera. Elastin levels were quantified with ImageJ (NIH, Bethesda, Mass.) from 4-5 regions per section. The same threshold was used for each photo to make sure they are comparable.

Western Blot Analysis: Protein samples from the thoracic aorta were prepared in lysis buffer as described previously. The proteins (40-50 mg) were resolved by SDS-PAGE and transferred to a nitrocellulose membrane (Bio-Rad). The membrane was then incubated overnight (4° C.) with a primary antibody against collagen-1, elastin, MMP2, MMP9, ALP (Abcam, 1:1000), TGFβ-1, TGFβ-3 (Santa Cruz, 1:100), RUX2, or α-Tubulin (Cell Signaling, 1:1000). Goat anti-mouse or goat anti-rabbit horseradish peroxidase (1:2000-1:5,000; Santa Cruz Biotechnology) was used as a secondary antibody and incubated for 1 hour at room temperature. Specific proteins were detected by chemiluminescent methods using Amersham™ ECL™ western blotting detection reagents (GE Healthcare, UK). Protein abundance on western blots was quantified by densitometry using Image lab software (Bio-Rad, Hercules, Calif.).

Reverse transcription-PCR (RT-PCR): Total RNA was extracted using a Direct-zol™ RNA Miniprep kit (Zymo Research, Irvine, Calif.) from kidney of adult and aged mice. The first-strand cDNA was synthesized from 500 ng of total RNA by using an iScript cDNA synthesis kit (Bio-Rad). The gene-specific primers for Klotho are shown in Table 1 in the parent application, U.S. Provisional Application No. 62/509,818.

The PCR conditions for the Klotho and α-Actin primers were as follows: hold for 5 min at 94° C., followed by 30 cycles consisting of denaturation at 94° C. (30 s), annealing at 57° C. (30 s), and elongation at 72° C. (1 min). The amplified products were subjected to electrophoresis on a 1% agarose gel. Each reaction was performed in triplicates. The gene expression was calculated as band intensity of Klotho/band intensity of a-Actin and plotted after normalization to the control group.

Methylation Analyses of Klotho Gene: The methylation status of CpG islands of mice Klotho gene was analysised using methylation-specific PCR (MSP) and bisulfite sequencing. First, the genomic DNA was extracted from kidney of adult and aged mice with tissue DNA Kit (Omega, Norlross, Ga.) and modified by bisulfate treatment (EZ DNA Methylation-Gold Kit, Zymo Research) for MSP analyses with gene promoter-specific primer pair that recognize the methylated and unmethylated CpG sites. The PCR product of genomic DNA without bisulfate treatment, with primers located in the promoter region of the mouse Klotho gene, was used as the inputted control for the MSP. The PCR products were visualized by ethidium bromide staining in 2% agarose gels, and the densitometric intensity corresponding to each band was quantified. Each reaction was performed in triplicates. The methylation index was calculated as (band intensity of MSP with methylated primers)/(band intensity of inputted control). The methylation index was plotted after normalization to the control group. The sequences of MSP primers and the amplification program are summarized in Table 2 in the parent application, U.S. Provisional Application No. 62/509,818. The PCR program conditions for the MSP primers are shown in Table 1 below.

TABLE 1
MSP primer amplification program

| | Program |
|---|---|
| Klotho set | 95° C. × 8 min;(95° C. × 1 min, 61° C. × 1 min, 72° C. × |
| | 2.5 min) × 40 cycles; 72° C. 5 min |
| Inputted | 95° C. × 8 min;(95° C. × 1 min, 59° C. × 1 min, 72° C. × |
| control | 2.5 min) × 40 cycles; 72° C. 5 min |
| (Klotho) | |

Measurement of DNA Demethylase and DNA Methyltransferase Activity: The DNA demethylase and DNA methyltransferase activity was measured by using DNA demethylase activity qualification kit (Abcam, Cambridge, Mass.) and methyltransferase colorimetric assay kit (Cayman Chemical Company, Ann Arbor, Mich.). Protein extracts from the thoracic aorta were prepare in lysis buffer. Assay procedures were followed by the manufacturer's protocols. The absorbance was read at 450 nm for DNA demethylase activity assay and 510 nm for DNA methyltransferase activity using BioTeck Multi-Mode Microplate Readers.

Measurement of MMP2 and MMP9 Activity: MMP2 and MMP9 activity were measured by Zymogram PAGE (Bio-Rad). Briefly, Lysates from thoracic aorta were quantified using BCA assay (Pierce) and prepared under non-reducing, non-denaturing conditions. Protein from lysates was separated on a zymogram gel containing Gelatin (Bio-Rad). After running, the gel was incubated in the Zymogram Renaturing Buffer with gentle agitation for 30 minutes at room temperature. Next, the gel was equilibrated in IX developing for 30 minutes on a shaker at room temperature. Then, fresh developing buffer was replaced and the gel was incubated at 37° C. overnight to develop. The next day, the gel was washed 3×, 5 minutes each, in doubly distilled water, then stained with brilliant blue R250 for 30 minutes. Gels are destained with an appropriate Coomassie R-250 destaining solution (Methanol:Acetic acid:Water (50:10:40). Areas of protease activity appear as clear bands against a dark blue background where the protease has digested the substrate.

Statistical Analysis: Quantitative data is presented as the Means±SE. Differences between experimental groups were examined by one-way analysis of variance (ANOVA) followed by the Bonferroni post-test using Prism software (GraphPad). For all analysis, p<0.05 were considered statistically significant.

Results

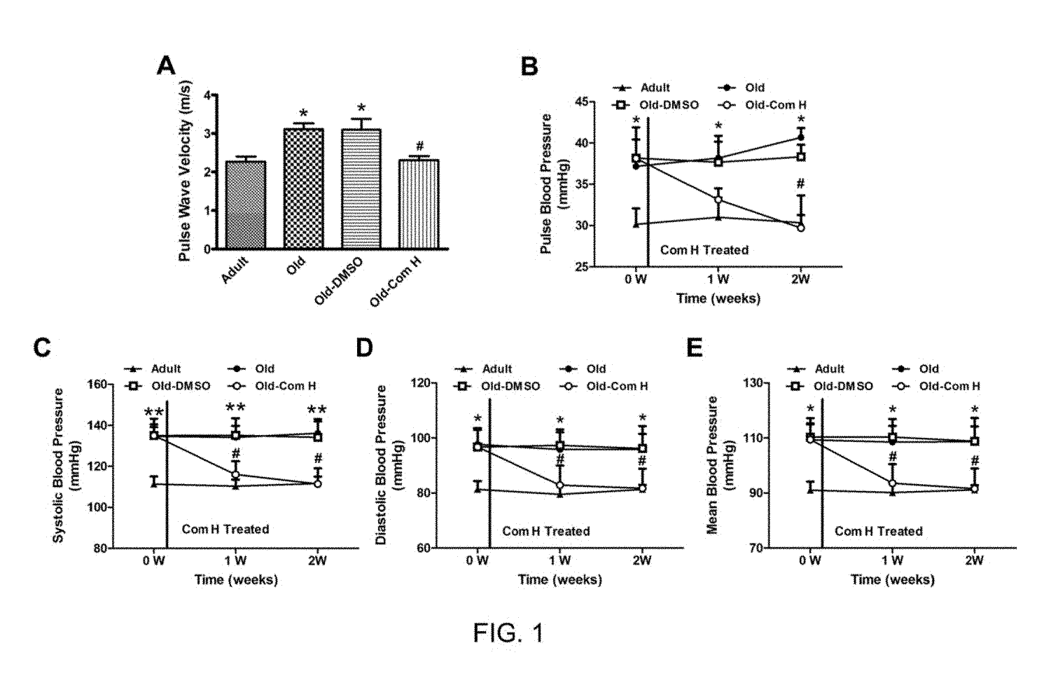

Activation of demethylase by the active agent attenuated arterial stiffness and hypertension in aged mice. Arterial pulse wave velocity (PWV) is a direct measure of arterial stiffness. The widening pulse pressure (the numeric difference between systolic and diastolic blood pressure) seen with aging is another direct indicator of arterial stiffness. PWV of aged mice was increased significantly compared to that of adult mice (3.11±0.15 m/s vs. 2.27±0.13 m/s, p<0.01) (FIG. 1A). Pulse pressure of aged mice was also significantly increased compared to that of adult mice in deferent time point (FIG. 1B), indicating that aging causes arterial stiffness. However, increasing circulating levels of Klotho by the active agent decreased PWV and pulse pressure after 2 week treatment in aged mice (FIGS. 1A and B). Besides reducing arterial stiffness, the active agent also decreased systolic blood pressure (FIG. 1C), diastolic blood pressure (FIG. 1D), and mean blood pressure (FIG. 1E) in aged mice to the adult levels after 2 weeks treatment. Therefore, increasing circulating levels of Klotho by the active agent reduced arterial stiffness and blood pressure in aged mice.

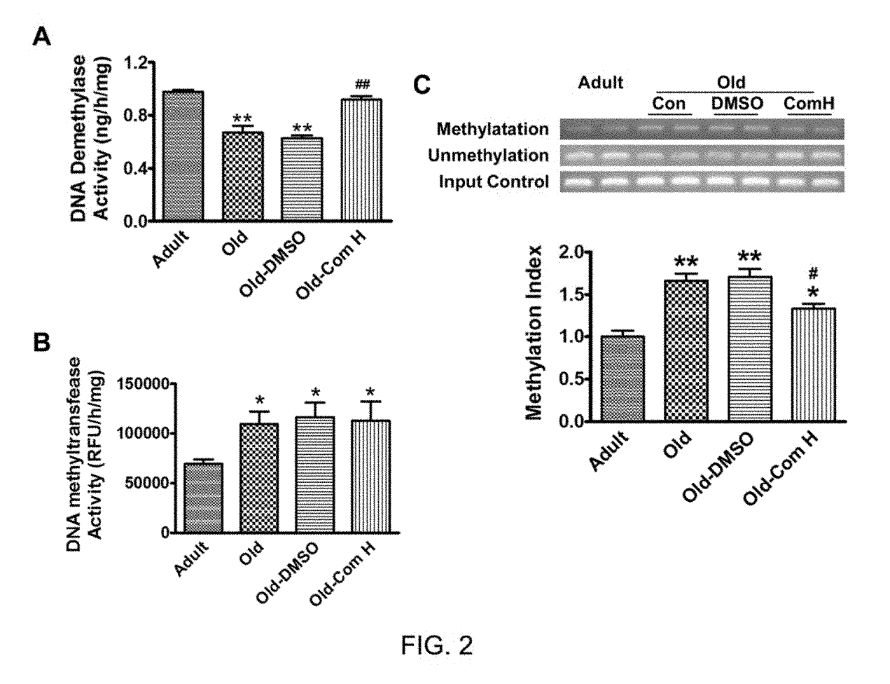

The active agent decreased the DNA hypermethylation of Klotho gene in aged mice. DNA demethylase activity and DNA methyltransferase activity was then measured. The DNA demethylase activity was significantly decreased and the DNA methyltransferase activity was significantly increased in aged mice (24-26 months old) compared to that of adult mice (12 months old) (FIGS. 2A and B). The active agent increased the DNA demethylase activity which was decreased in aged mice, while the active agent did not affect the DNA methyltransferase activity (FIGS. 2A and B). The methylation of the Klotho gene was then measured. The methylation of Klotho gene was significantly increased in aged mice compared to that of adult mice (FIG. 2C). Thus, the active agent inhibited the methylation of Klotho gene which was increased in aged mice (FIG. 2C).

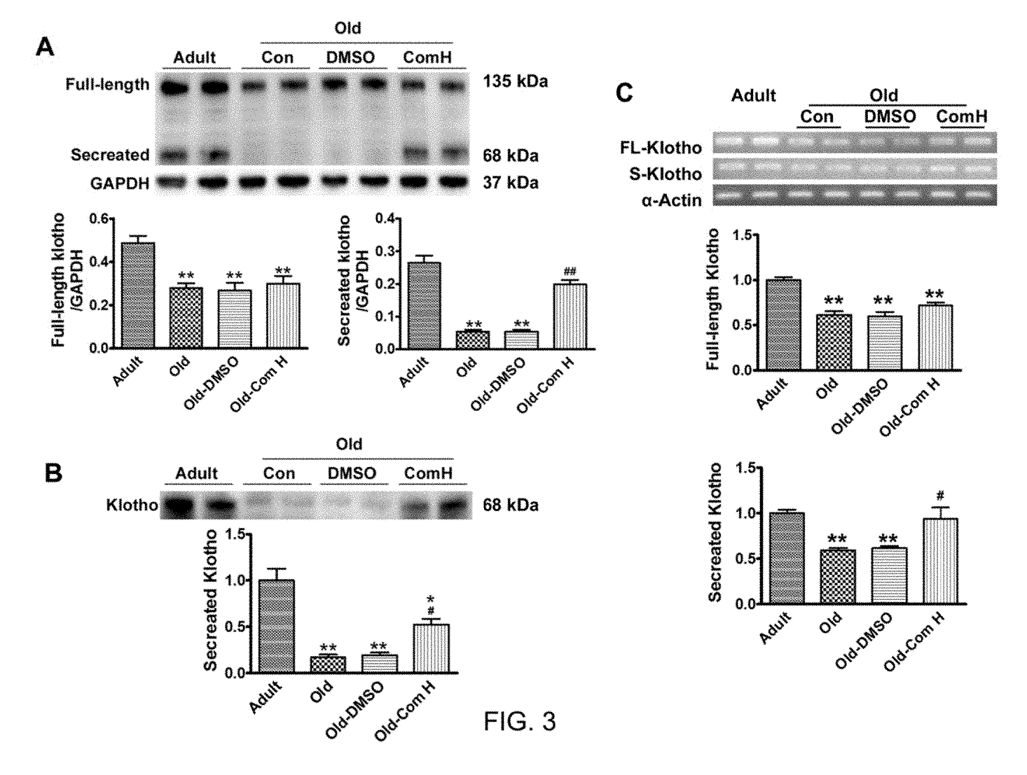

The active agent increased expression of secreted Klotho in aged mice. Secreted Klotho, acting as a hormone, shows different functions from full-length Klotho. The active agent significantly increased secreted Klotho protein expression in kidney of 24-26 month-old mice, while the active agent did not affect full-length Klotho expression (FIG. 3A). The circulating levels of secreted Klotho were also markedly increased by the active agent (FIG. 3B). Besides protein expression, the active agent also increased secreted Klotho mRNA expression in kidney (FIG. 3C). Taken together, the active agent increased secreted Klotho mRNA and protein expression in aged mice.

Further, increasing circulating levels of Klotho by the active agent prevented accumulation of stiffer collagen and degeneration of compliant elastin fibers in aortas of aged mice. The arterial collagen and elastin levels were measured by immunostaining and western blot assays. The immunostaining assay showed that aortic collagen levels were increased significantly in aged mice (FIG. 4A). Aging-induced collagen deposition was mainly found in the medial and adventitial layer of the aorta. Aortic elastin levels were decreased significantly in aged mice (FIG. 4A). Western blot analysis confirmed that aging upregulated collagen I expression but downregulated elastin levels in aortas (FIG. 4B). The ratio of elastin to collagen in aortas was markedly decreased in aged mice (FIGS. 4A-4B), indicating that aging causes arterial remodeling. Due to increasing circulating levels of Klotho, the active agent abolished upregulation of collagen and downregulation of elastin in aortas leading to attenuation of arterial remodeling in aged mice (FIGS. 4A-4B). The active agent effectively decreased collagen production and increased elastin production (as shown in FIG. 4B), indicating that it is an effective antiaging treatment for providing arterial remodeling.

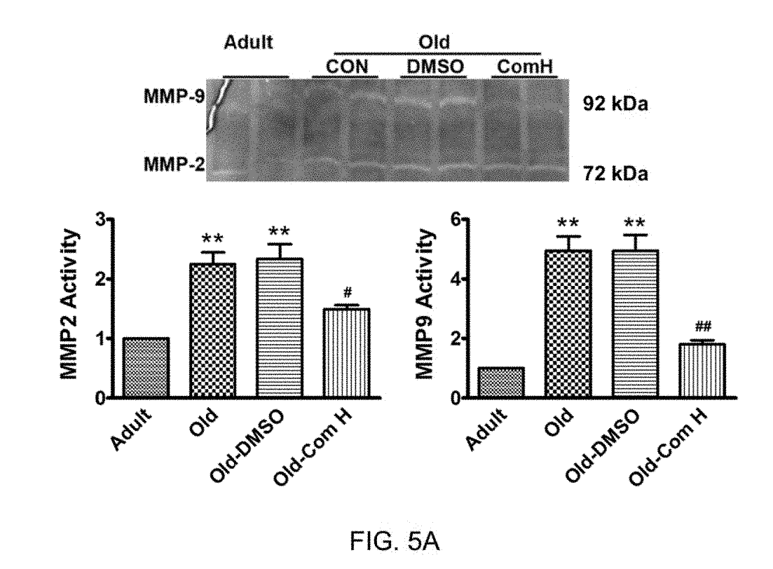

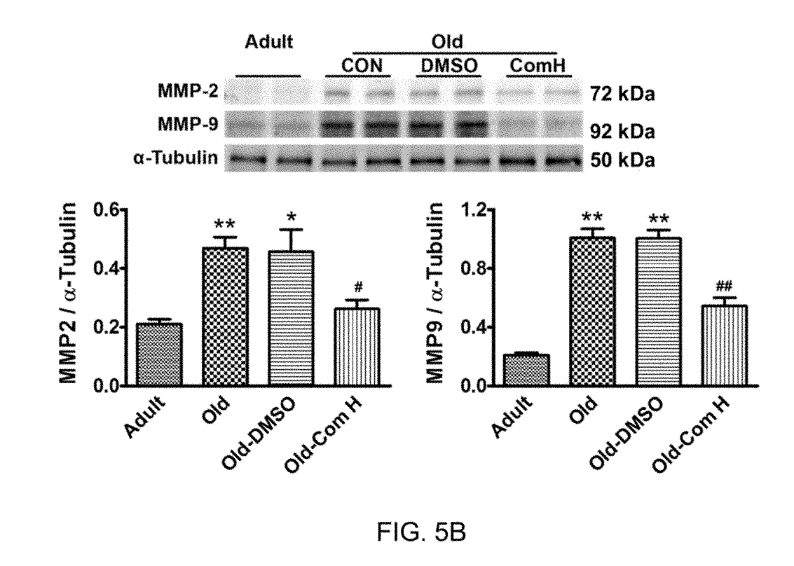

Aging increased arterial MMP activity and expression, which can be eliminated by the active agent. MMPs are a family of proteases that play important roles in extracellular matrix (ECM) remodeling and degradation. Increased MMP activity contributes to ECM remodeling and fibrosis. MMPs activity was measured by zymography. MMP2 and MMP9 activities were increased significantly in aortas of aged mice (FIG. 5A). Increasing circulating levels of Klotho by the active agent decreased MMP2 and MMP9 activities to the control levels of adult mice (FIG. 5A). MMPs expression levels were measured by western blot. MMP2 and MMP9 protein expressions were increased significantly in aortas of aged mice (FIG. 5B). Increasing circulating levels of Klotho by the active agent decreased MMP2 and MMP9 expressions to the control levels (FIG. 5B).

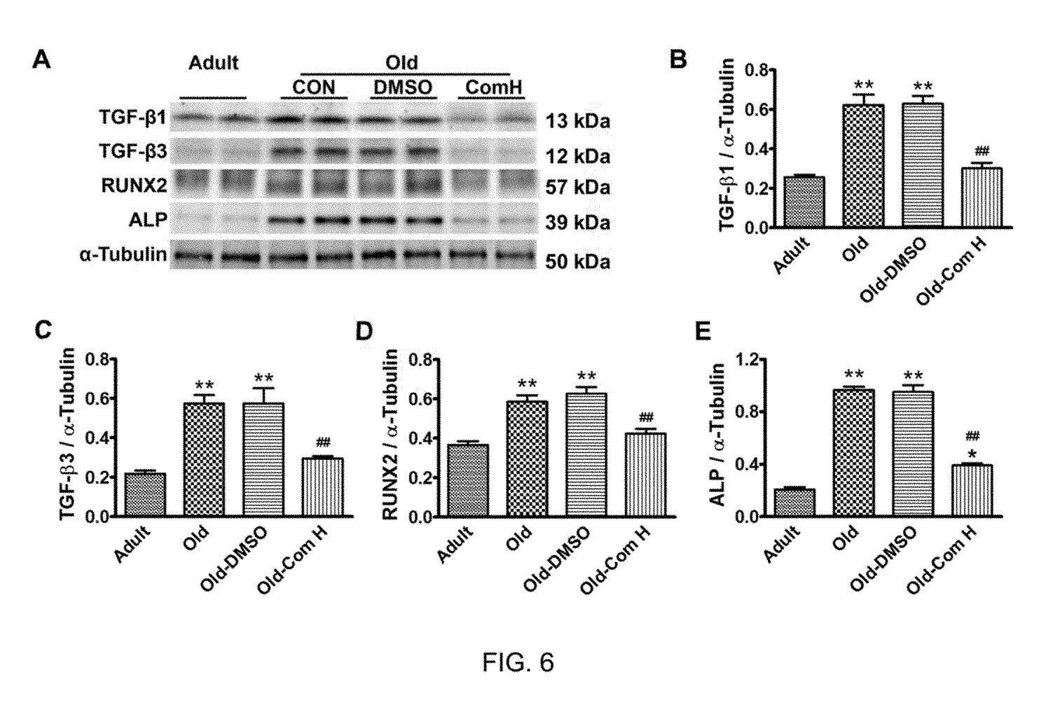

Aging increased arterial TGF-β1, TGF-β3, RUNX2 and ALP expression, which can be abolished by the active agent. TGFβ increases matrix protein synthesis and decreases matrix protein degradation, resulting in tissue fibrosis. Runt-related transcription factor 2 (RUNX2) and Alkaline phosphatase (ALP) are another two markers of fibrosis and arterial stiffening. Western blot analysis showed that TGFβ1, TGF-β3, RUNX2 and ALP expressions were increased significantly in the aortas of aged mice (FIG. 6A-E). Increasing circulating levels of Klotho by the active agent decreased TGFβ1, TGF-β3, RUNX2 and ALP expressions to the control levels of adult mice (FIG. 6A-E), indicating that aging-induced upregulation of TGFβ1, TGF-β3, RUNX2 and ALP can be abolished by increasing circulating levels of Klotho.

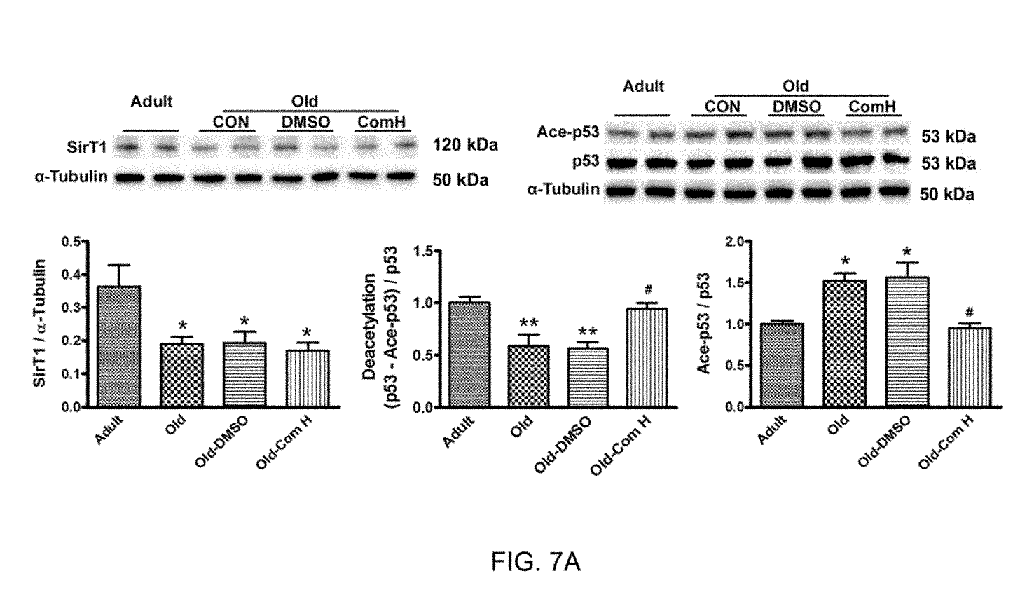

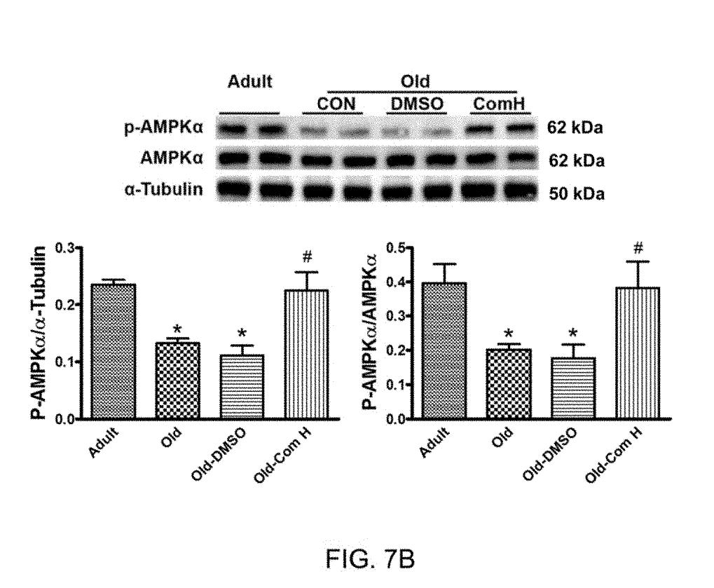

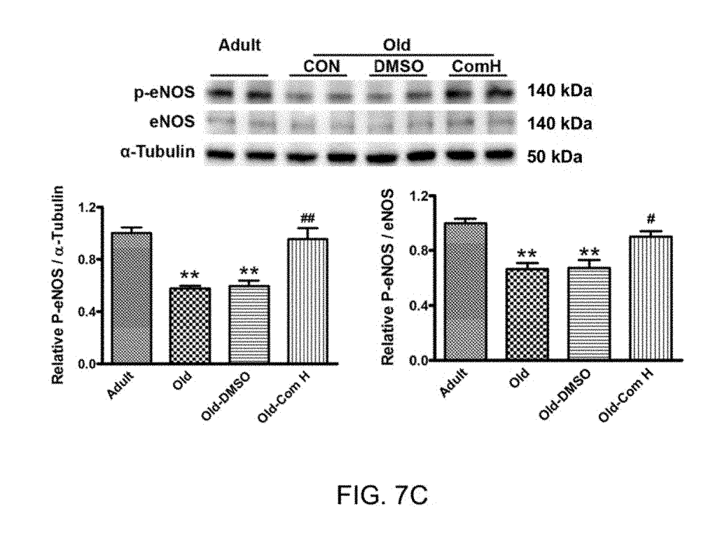

Aging inhibited SIRT1-AMPK-eNOS pathway which can be activated by the active agent. Silent information regulator T1 (SIRT1) plays an important role in the regulation of aging and longevity in mammals. The cross-talks between SIRT1 and AMP-activated protein kinase (AMPK), both activity eNOS activity, is indicated for controlling the senescence program. Therefore, the SIRT1-AMPK-eNOS cascade changing in aged mice and the effect of the active agent on this cascade was measured. The SIRT1 expression and activity in the aortas of aged mice were decreased significantly compared to adult mice. Although it did not affect SIRT1 expression, the active agent increased SIRT1 activity significantly, as evidenced by an increase in deacetylation of p53 tumor suppressor protein (FIG. 7A). The phospho-AMPK and phospho-eNOS expression were also decreased in the aortas of aged mice, indicated AMPK and eNOS activity were decreased. However, the active agent increased AMPK and eNOS activity in the aortas of aged mice (FIGS. 7B and 7C). Therefore, the SIRT1-AMPK-eNOS cascade pathway was suppressed in the aortas of aged mice, which can be activated by the active agent.

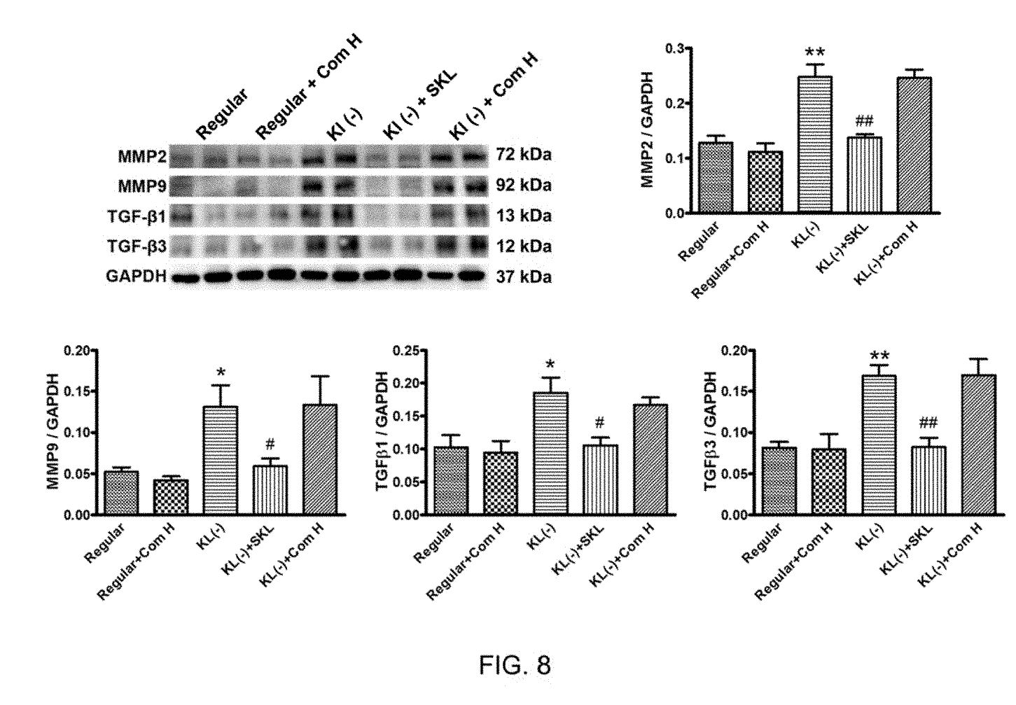

The active agent did not affect MMP2, MMP9, TGFβ1, and TGFβ3 expression in mouse vascular aortic smooth muscle cells (MOVAS). The in vivo example showed that the active agent increased circulating levels of Klotho and attenuated arterial stiffening and hypertension in aged mice. Without wishing to be bound by theory, the results demonstrate that the active agent attenuates arterial stiffening and hypertension through increasing circulating levels of Klotho. Mouse aortic smooth muscle cells (MOVAS) which do not express endogenous Klotho were treated with active agent for 16 h and then harvested for western blot analysis. In regular medium, which has constant levels of Klotho in the serum, active agent treatment did not change MMP2, MMP9, TGFβ1, and TGFβ3 expressions (FIG. 8), indicating that active agent did not have direct effect on MMP2, MMP9, TGFβ1, and TGFβ3 expressions in smooth muscle cells. In Klotho free medium (KL(−)) treated cells, MMP2, MMP9, TGFβ1, and TGFβ3 expressions were increased significantly compared to that of regular medium treated cells (FIG. 8), indicating that Klotho deficiency directly upregulates MMP2, MMP9, TGFβ1, and TGFβ3 expressions. However, secreted Klotho (sKL) treatment significantly decreased MMP2, MMP9, TGFβ1, and TGFβ3 expressions (FIG. 8), indicating that Klotho directly regulates MMP2, MMP9, TGFβ1, and TGFβ3 expressions. But the active agent treatment did not affect MMP2, MMP9, TGFβ1, and TGFβ3 expressions in Klotho free medium treated cells (FIG. 8). These results indicate that Klotho decreases MMP2, MMP9, TGFβ1, and TGFβ3 expressions to inhibit arterial fibrosis and stiffness, and active agent has no direct effect on arterial fibrosis and stiffness without increasing Klotho expression. Therefore, the active agent attenuates arterial stiffening and hypertension through increasing circulating levels of Klotho. Another use of the active agent is an antiaging treatment for providing arterial remodeling which occurs as a result of a decrease in collagen production and an increase in elastin production (as shown in FIG. 4B).

### DISCUSSION

Aging is defined as the age-related decline in physiological function essential for survival and fertility. Age can result in cardiovascular outcomes, such as arterial stiffness and hypertension. The results from the above described example, show that blood pressure (BP) and pulse wave velocity (PWV) were increased in 24-27 month-old mice, with the age of equivalent of 70-80 years in humans. This demonstrates that activation of demethylase by the active agent reversed arterial stiffening and hypertension in aged mice (FIG. 1), indicating that increased methylation contributes to aging-related arterial stiffening and hypertension, and demonstrating that the active agent is an effective therapeutic agent for arterial stiffness and hypertension. Another use of the active agent is as an anti-aging treatment for providing arterial remodeling which occurs as a result of a decrease in collagen production and an increase in elastin production (as shown in FIG. 4B).

DNA methylation is one of several epigenetic mechanisms that cells use to control gene expression. During development, the pattern of DNA methylation in the genome changes as a result of a dynamic process involving both DNA methylation and demethylation. The methylation state of the promoter region is related to Klotho mRNA expression, indicating that Klotho expression is regulated by DNA methylation. The above described example shows that DNA demethylase activity was decreased and the DNA methyltransferase activity was increased in aged mice (FIG. 2), indicating that aging increases DNA methylation.

DNA methylation of Klotho gene was increased in aged mice compared to that of adult mice, which was decreased by the active agent (FIG. 2). The active agent increased the DNA demethylase activity which was decreased in aged mice, while it did not affect the DNA methyltransferase activity. The active agent increased Klotho expression in aged mice through increasing the DNA demethylase activity to decrease the hypermethylation of Klotho gene. Therefore, the attenuation of aging-related arterial stiffening and hypertension by the active agent is attributed, at least in part, to increased Klotho levels. The example described herein shows that deficiency of Klotho gene caused arterial stiffness and hypertension. As discussed above, at age 70 years, the serum level of Klotho in a human is only about one half of what it was at age 40 years, while the prevalence of arterial stiffness and hypertension increases with age. Thus, Klotho deficiency is a pathological factor for aging-associated arterial stiffness and hypertension.

Three types of Klotho protein with potentially different functions have been identified: (1) a full-length transmembrane Klotho; (2) a truncated soluble Klotho; and (3) a secreted Klotho. The full-length Klotho is mainly expressed in kidney distal tubule cells and serves as a co-receptor of FGF23 and enhances FGF23 signaling to maintain mineral metabolism. While the circulating Klotho, including soluble Klotho and secreted Klotho, may act as a hormone and regulate the functions in tissues or cells that do not express Klotho (e.g., vascular endothelial cells and smooth muscle cells). In this example, both full-length and secreted Klotho were decreased in aged mice. The active agent increased secreted Klotho but not the transmembrane form of Klotho.

The in vivo example shows that the active agent increased circulating levels of Klotho and attenuated arterial stiffening and hypertension in aged mice, indicating that active agent attenuates arterial stiffening and hypertension through increasing circulating levels of Klotho. Mouse aortic smooth muscle cells (MOVAS) which do not express endogenous Klotho were treated with active agent and some arterial stiffness related protein expression was measured by western blot analysis. Because of no endogenous Klotho expression in MOVAS, the active agent did not affect MMP2, MMP9, TGFβ1, and TGFβ3 expressions in either Klotho-contained medium or Klotho free medium. However, Klotho itself affects MMP2, MMP9, TGFβ1, and TGFβ3 expressions. Without wishing to be bound by theory, these results indicate that Klotho decreases MMP2, MMP9, TGFβ1, and TGFβ3 expressions to inhibit arterial fibrosis and stiffness, and that the active agent has no direct effect on arterial fibrosis and stiffness without increasing Klotho expression. Based on the results of the in vivo and in vitro examples discussed above, the active agent attenuates arterial stiffening and hypertension through increasing circulating levels of Klotho. Another use of the active agent is an antiaging treatment for providing arterial remodeling which occurs as a result of a decrease in collagen production and an increase in elastin production (as shown in FIG. 4B).

The results of the example show that SIRT1 activity was decreased in aged mice, while the active agent increased SIRT1 activity. SIRT1, known as class III histone deacetylases, is a nuclear protein implicated in the regulation of many cellular processes, including apoptosis, cellular senescence, endocrine signaling, glucose homeostasis, aging, and longevity. SIRT1 has been reported to deacetylate the lysine residues of a number of nuclear proteins, such as p53, NF-κB, PGC-1a, CBP/p300, and forkhead family proteins. Sirt1 can inhibit TGF-β signaling and ameliorate fibrosis. In this example, aging decreased SIRT1 activity, increased TGFβ and MMP expression, and induced fibrosis. So, the inhibition of SIRT1 activity is the initial cause of aging-induced fibrosis.

Exposure to the active agent decreased blood pressure after only 1 week of treatment. This acute effect is due to endothelium protection. Western blot results showed that the expression of peNOS and pAMPK is decreased in aged mice, while the active agent increased their expression. Endothelial nitric-oxide synthase (eNOS) is an important enzyme in the cardiovascular system. It catalyzes the production of nitric oxide (NO), a key regulator of blood pressure, vascular remodeling, and angiogenesis. Several protein kinases including Akt/PKB, PKA, and AMPK activate eNOS by phosphorylating Ser1177 in response to various stimuli. Due to increasing pAMPK and peNOS expression, the active agent induces more NO production to protect endothelial function to low blood pressure.

While the present disclosure has been described herein in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure as defined herein. Thus the examples described above, which include particular embodiments, will serve to illustrate the practice of the inventive concepts of the present disclosure, it being understood that the particulars shown are by way of example and for purposes of illustrative discussion of particular embodiments only and are presented in the cause of providing what is believed to be the most useful and readily understood description of procedures as well as ofthe principles and conceptual aspects of the present disclosure. Changes may be made in the formulation of the various compositions described herein, the methods described herein or in the steps or the sequence of steps of the methods described herein without departing from the spirit and scope of the present disclosure. Further, while various embodiments of the present disclosure have been described in claims herein below, it is not intended that the present disclosure be limited to these particular claims.

### REFERENCES

The following references, to the extent that they provide exemplary procedural or other details supplementary to those set forth herein, are specifically incorporated herein by reference.
Avrahami D, Li C, Zhang J, Schug J, Avrahami R, Rao S, Stadler M B, Burger L, Schubeler D, Glaser B, Kaestner K H. Aging-Dependent Demethylation of Regulatory Elements Correlates with Chromatin State and Improved beta Cell Function. Cell Metab. 2015; 22 (4): 619-632.
Wagner W, Femandez-Rebollo E, Frobel J. DNA-methylation changes in replicative senescence and aging: two sides of the same coin? Epigenomics. 2016; 8 (1): 1-3.
Zinovkina L A, Zinovkin R A. DNA Methylation, Mitochondria, and Programmed Aging. Biochemistry (Mosc). 2015; 80 (12): 1571-1577.
Bigot A, Duddy W J, Ouandaogo Z G, Negroni E, Mariot V, Ghimbovschi S, Harmon B, Wielgosik A, Loiseau C, Devaney J, Dumonceaux J, Butler-Browne G, Mouly V, Duguez S. Age-Associated Methylation Suppresses SPRY 1, Leading to a Failure of Re-quiescence and Loss of the Reserve Stem Cell Pool in Elderly Muscle. Cell Rep. 2015; 13 (6): 1172-1182.
Alvares S M, Mayberry G A, Joyner E Y, Lakowski B, Ahmed S. H3K4 demethylase activities repress proliferative and postmitotic aging. Aging Cell. 2014; 13 (2): 245-253.
Sun Z. Aging, arterial stiffness, and hypertension. Hypertension. 2015; 65 (2): 252-256.
Hashimoto J, Ito S. Central pulse pressure and aortic stiffness determine renal hemodynamics: pathophysiological implication for microalbuminuria in hypertension. Hypertension. 2011; 58 (5): 839-846.
Hashimoto J, Ito S. Aortic stiffness determines diastolic blood flow reversal in the descending thoracic aorta: potential implication for retrograde embolic stroke in hypertension. Hypertension. 2013; 62 (3): 542-549.
Karras A, Haymann J P, Bozec E, Metzger M, Jacquot C, Maruani G, Houillier P, Froissart M, Stengel B, Guardiola P, Laurent S, Boutouyrie P, Briet M, Nephro Test Study G. Large artery stiffening and remodeling are independently associated with all-cause mortality and cardiovascular events in chronic kidney disease. Hypertension. 2012; 60 (6): 1451-1457.
Kitzman D W, Herrington D M, Brubaker P H, Moore J B, Eggebeen J, Haykowsky M J. Carotid arterial stiffness and its relationship to exercise intolerance in older patients with heart failure and preserved ejection fraction. Hypertension. 2013; 61 (1): 112-119.
Kuro-o M. Klotho in health and disease. Current opinion in nephrology and hypertension. 2012; 21 (4): 362-368.
Kuro-o M, Matsumura Y, Aizawa H, Kawaguchi H, Suga T, Utsugi T, Ohyama Y, Kurabayashi M, Kaname T, Kume E, Iwasaki H, lida A, Shiraki-lida T, Nishikawa S, Nagai R, Nabeshima Y I. Mutation of the mouse klotho gene leads to a syndrome resembling ageing. Nature. 1997; 390 (6655): 45-51.
Kurosu H, Yamamoto M, Clark J D, Pastor J V, Nandi A, Gurnani P, McGuinness O P, Chikuda H, Yamaguchi M, Kawaguchi H, Shimomura 1, Takayama Y, Herz J, Kahn C R, Rosenblatt K P, Kuro-o M. Suppression of aging in mice by the hormone Klotho. Science. 2005; 309 (5742): 1829-1833.
Zhou L, Li Y, Zhou D, Tan R J, Liu Y. Loss of Klotho contributes to kidney injury by derepression of Wnt/beta-catenin signaling. Journal of the American Society of Nephrology: JASN. 2013; 24 (5): 771-785.
Lin Y, Sun Z. In vivo pancreatic beta-cell-specific expression of antiaging gene Klotho: a novel approach for preserving beta-cells in type 2 diabetes. Diabetes. 2015; 64 (4): 1444-1458.
Doi S, Zou Y, Togao O, Pastor J V, John G B, Wang L, Shiizaki K, Gotschall R, Schiavi S, Yorioka N, Takahashi M, Boothman D A, Kuro-o M. Klotho inhibits transforming growth factor-beta1 (TGF-beta1) signaling and suppresses renal fibrosis and cancer metastasis in mice. The Journal of biological chemistry. 2011; 286 (10): 8655-8665.
Moe S M. Klotho: a master regulator of cardiovascular disease? Circulation. 2012; 125 (18): 2181-2183.
Chen K, Zhou X, Sun Z. Haplodeficiency of Klotho Gene Causes Arterial Stiffening via Upregulation of Scleraxis Expression and Induction of Autophagy. Hypertension. 2015; 66 (5): 1006-1013.
Xiao N M, Zhang Y M, Zheng Q, Gu J. Klotho is a serum factor related to human aging. Chinese medical journal. 2004; 117 (5): 742-747.
Kotsis V, Stabouli S. Arterial stiffness, vascular aging, and intracranial large artery disease. American journal of hypertension. 2011; 24 (3): 252.
Karras A, Haymann J P, Bozec E, Metzger M, Jacquot C, Maruani G, Houillier P, Froissart M, Stengel B, Guardiola P, Laurent S, Boutouyrie P, Briet M. Large artery stiffening and remodeling are independently associated with all-cause mortality and cardiovascular events in chronic kidney disease. Hypertension. 2012; 60 (6): 1451-1457.
Hartley C J, Taffet G E, Michael L H, Pham T T, Entman M L. Noninvasive determination of pulse-wave velocity in mice. The American journal of physiology. 1997; 273 (1 Pt 2): H494-500.
Steppan J, Barodka V, Berkowitz D E, Nyhan D. Vascular stiffness and increased pulse pressure in the aging cardiovascular system. Cardiology research and practice. 2011; 2011: 263585.
Capelli A, Lusuardi M, Cerutti C G, Donner C F. Lung alkaline phosphatase as a marker of fibrosis in chronic interstitial disorders. American journal of respiratory and critical care medicine. 1997; 155 (1): 249-253.
Lin M E, Chen T, Leaf E M, Speer M Y, Giachelli C M. Runx2 Expression in Smooth Muscle Cells Is Required for Arterial Medial Calcification in Mice. The American journal of pathology. 2015; 185 (7): 1958-1969.
Wang F, Chen H Z, Lv X, Liu D P. SIRT1 as a novel potential treatment target for vascular aging and age-related vascular diseases. Current molecular medicine. 2013; 13 (1): 155-164.
Satoh A, Brace C S, Rensing N, Cliften P, Wozniak D F, Herzog E D, Yamada K A, Imai S. Sirt1 extends life span and delays aging in mice through the regulation of Nk2 homeobox 1 in the DMH and LH. Cell metabolism. 2013; 18 (3): 416-430.
Alcendor R R, Gao S, Zhai P, Zablocki D, Holle E, Yu X, Tian B, Wagner T, Vatner S F, Sadoshima J. Sirt1 regulates aging and resistance to oxidative stress in the heart. Circulation research. 2007; 100 (10): 1512-1521.
Wang Y, Liang Y, Vanhoutte P M. SIRT1 and AMPK in regulating mammalian senescence: a critical review and a working model. FEBS letters. 2011; 585 (7): 986-994.
Flurkey K C J, Harrison D E. The Mouse in Aging Research. The Mouse in Biomedical Research 2nd Edition. 2007: 637-672.
Moore L D, Le T, Fan G. DNA methylation and its basic function. Neuropsychopharmacology: official publication of the American College of Neuropsychopharmacology. 2013; 38 (1): 23-38.
Azuma M, Koyama D, Kikuchi J, Yoshizawa H, Thasinas D, Shiizaki K, Kuro-o M, Furukawa Y, Kusano E. Promoter methylation confers kidney-specific expression of the Klotho gene. FASEB journal: official publication of the Federation of American Societies for Experimental Biology. 2012; 26 (10): 4264-4274.
King G D, Rosene D L, Abraham C R. Promoter methylation and age-related downregulation of Klotho in rhesus monkey. Age. 2012; 34 (6): 1405-1419.
Sun C Y, Chang S C, Wu M S. Suppression of Klotho expression by protein-bound uremic toxins is associated with increased DNA methyltransferase expression and DNA hypermethylation. Kidney international. 2012; 81 (7): 640-650.
Young G H, Wu V C. KLOTHO methylation is linked to uremic toxins and chronic kidney disease. Kidney international. 2012; 81 (7): 611-612.
Chen K, Zhou X, Sun Z. Haplodeficiency of Klotho Gene Causes Arterial Stiffening via Upregulation of Scleraxis Expression and Induction of Autophagy. Hypertension. 2015.
Xu Y, Sun Z. Molecular basis of Klotho: from gene to function in aging. Endocrine reviews. 2015; 36 (2): 174-193.
Yuan F, Xie Q, Wu J, Bai Y. Mao B, Dong Y, Bi W, Ji G, Tao W, Wang Y, Yuan Z. MSTI promotes apoptosis through regulating Sirt1-dependent p53 deacetylation. The Journal of biological chemistry. 2011; 286 (9): 6940-6945.
Salminen A, Kaarniranta K. NF-kappaB signaling in the aging process. Journal of clinical immunology. 2009; 29 (4): 397-405.
Amat R, Planavila A, Chen S L, Iglesias R, Giralt M, Villarroya F. SIRT1 controls the transcription of the peroxisome proliferator-activated receptor-gamma Co-activator-1alpha (PGC-1alpha) gene in skeletal muscle through the PGC-1alpha autoregulatory loop and interaction with MyoD. The Journal of biological chemistry. 2009; 284 (33): 21872-21880.
Das C, Lucia M S, Hansen K C, Tyler J K. CBP/p300-mediated acetylation of histone H3 on lysine 56. Nature. 2009; 459 (7243): 113-117.
Brunet A, Sweeney L B, Sturgill J F, Chua K F, Greer P L, Lin Y, Tran H, Ross S E, Mostoslavsky R, Cohen H Y, Hu L S, Cheng H L, Jedrychowski M P, Gygi S P, Sinclair D A, Alt F W, Greenberg M E. Stress-dependent regulation of FOXO transcription factors by the SIRT1 deacetylase. Science. 2004; 303 (5666): 2011-2015.
Kume S, Haneda M, Kanasaki K, Sugimoto T, Araki S, Isshiki K, Isono M, Uzu T, Guarente L, Kashiwagi A, Koya D. SIRT1 inhibits transforming growth factor beta-induced apoptosis in glomerular mesangial cells via Smad7 deacetylation. The Journal of biological chemistry. 2007; 282 (1): 151-158.
Zerr P, Palumbo-Zerr K, Huang J, Tomcik M, Sumova B, Distler O, Schett G, Distler J H. Sirt1 regulates canonical TGF-beta signalling to control fibroblast activation and tissue fibrosis. Annals of the rheumatic diseases. 2014.
Huang X Z, Wen D, Zhang M, Xie Q, Ma L, Guan Y, Ren Y, Chen J, Hao C M. Sirt1 activation ameliorates renal fibrosis by inhibiting the TGF-beta/Smad3 pathway. Journal of cellular biochemistry. 2014; 115 (5): 996-1005.
Shaul P W. Regulation of endothelial nitric oxide synthase: location, location, location. Annual review of physiology. 2002; 64: 749-774.
Dimmeler S, Fleming I, Fisslthaler B, Hermann C, Busse R, Zeiher A M. Activation of nitric oxide synthase in endothelial cells by Akt-dependent phosphorylation. Nature. 1999; 399 (6736): 601-605.
Fulton D, Gratton J P, McCabe T J, Fontana J, Fujio Y, Walsh K, Franke T F, Papapetropoulos A, Sessa W C. Regulation of endothelium-derived nitric oxide production by the protein kinase Akt. Nature. 1999; 399 (6736): 597-601.
Hartley C J, Taffet G E, Michael L H, Pham T T, Entman M L. Noninvasive determination of pulse-wave velocity in mice. The American journal of physiology. 1997; 273: H494-500.
Reddy A K, Li Y H, Pham T T, Ochoa L N, Trevino M T, Hartley C J, Michael L H, Entman M L, Taffet G E. Measurement of aortic input impedance in mice: Effects of age on aortic stiffness. American journal of physiology. Heart and circulatory physiology. 2003; 285: H1464-1470.
Whitesall S E, Hoff J B, Vollmer A P, D'Alecy L G. Comparison of simultaneous measurement of mouse systolic arterial blood pressure by radiotelemetry and tail-cuff methods. Am J Physiol Heart Circ Physiol. 2004; 286: H2408-2415.
Feng M, Whitesall S, Zhang Y, Beibel M, D'Alecy L, DiPetrillo K. Validation of volume-pressure recording tail-cuff blood pressure measurements. Am J Hypertens. 2008; 21: 1288-1291.
Wang X, Skelley L, Wang B, Mejia A, Sapozhnikov V, Sun Z. Aav-based rnai silencing of nadph oxidase gp91 (phox) attenuates cold-induced cardiovascular dysfunction. Hum. Gene Ther. 2012; 23: 1016-1026.
Chen K, Kobayashi S, Xu X, Viollet B, Liang Q. Amp activated protein kinase is indispensable for myocardial adaptation to caloric restriction in mice. PloS one. 2013; 8: e59682.
Sun C Y, Chang S C, Wu M S. Suppression of klotho expression by protein-bound uremic toxins is associated with increased DNA methyltransferase expression and DNA hypermethylation. Kidney international. 2012; 81: 640-650.

What is claimed is:

1. A method of treating at least one of arterial stiffness, hypertension in a subject in need of such therapy, comprising:
administering to the subject an effective amount of a compound comprising an N-halophenyl-1H-indole-3-carboxamide, or a pharmaceutically-acceptable salt thereof, wherein the N-halophenyl-1H-indole-3-carboxamide has a halogen on at least one of the 2, 3, 4, 5, and 6-carbon positions of the phenyl ring, wherein the halogen is selected from the group consisting of chlorine (Cl), fluorine (F), bromine (Br), and iodine (I).

2. The method of claim 1, wherein the N-halophenyl-1H-indole-3-carboxamide or pharmaceutically-acceptable salt thereof is represented by Formula I: 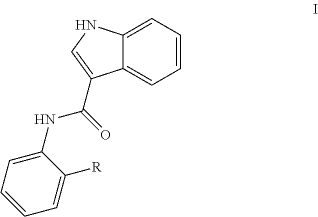
wherein R is selected from the group consisting of Cl, F, Br, and I.

3. The method of claim 2, wherein R is Cl.

4. A method of treating arterial aging in a subject in need of such therapy, comprising:
administering to the subject an effective amount of a compound that increases arterial elastin production, the compound comprising an N-halophenyl-1H-indole-3-carboxamide, or a pharmaceutically-acceptable salt thereof, wherein the N-halophenyl-1H-indole-3-carboxamide has a halogen on at least one of the 2, 3, 4, 5, and 6-carbon positions of the phenyl ring, wherein the halogen is selected from the group consisting of chlorine (Cl), fluorine (F), bromine (Br), and iodine (1).

5. The method of claim 4, wherein the N-halophenyl-1H-indole-3-carboxamide or pharmaceutically-acceptable salt thereof is represented by Formula I: 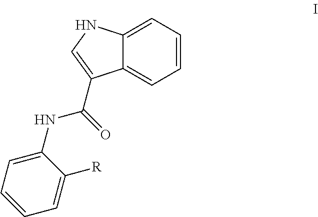
wherein R is selected from the group consisting of Cl, F, Br, and I.

6. The method of claim 5, wherein R is Cl.

7. The method of claim 4, wherein the compound decreases arterial collagen production.

8. A method of treating diabetes or a diabetes-related disease or condition in a subject in need of such therapy, comprising:
administering to the subject an effective amount of a compound comprising an N-halophenyl-1H-indole-3-carboxamide, or a pharmaceutically-acceptable salt thereof, wherein the N-halophenyl-1H-indole-3-carboxamide has a halogen on at least one of the 2, 3, 4, 5, and 6-carbon positions of the phenyl ring, wherein the halogen is selected from the group consisting of chlorine (Cl), fluorine (F), bromine (Br), and iodine (I).

9. The method of claim 8, wherein the N-halophenyl-1H-indole-3-carboxamide or pharmaceutically-acceptable salt thereof is represented by Formula I: 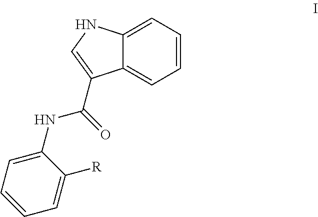
wherein R is selected from the group consisting of Cl, F, Br, and I.

10. The method of claim 9, wherein R is Cl.

11. The method of claim 8, wherein the diabetes or diabetes-related disease or condition is selected from the group consisting of Type 1 diabetes mellitus (T1DM), Type 2 diabetes mellitus (T2DM), hyperinsulenima, obesity; peripheral arterial disease (PAD) of the arms, legs, and feet; foot ulcers; diabetic neuropathy; diabetic retinopathy; diabetic kidney disease; ketoacidosis; and hyperosmolar hyperglycemic nonketotic syndrome (HHNS).
